SEQUENCE LISTING (2 sequences)

SEQ ID NO: 1 - DNA, 2772 bp, Homo sapiens
  human memin, multiple endocrine neoplasia-type 1 (MEN1) cDNA
ggtgtccggagccgcggacctagagatcccagaagccacagcgcagcggcccggcccgccactatttccaggctctgcggggcaggggccgccgcccaccgcccgccgccatggggctgaaggccgcccagaagacgctgttcccgctgcgctccatcgacgacgtggtgcgcctgtttgctgccgagctgggccgagaggagccggacctggtgctcctttccttggtgctgggcttcgtggagcattttctggctgtcaaccgcgtcatccctaccaacgttcccgagctcaccttccagcccagccccgcccccgacccgcctggcggcctcacctactttcccgtggccgacctgtctatcatcgccgccctctatgcccgcttcaccgcccagatccgaggcgccgtcgacctgtccctctatcctcgagaagggggtgtctccagccgtgagctggtgaagaaggtctccgatgtcatatggaacagcctcagccgctcctacttcaaggatcgggcccacatccagtccctcttcagcttcatcacaggcaccaaattggacagctccggtgtggcctttgctgtggttggggcctgccaggccctgggtctccgggatgtccacctcgccctgtctgaggatcatgcctgggtagtgtttgggcccaatggggagcagacagctgaggtcacctggcacggcaagggcaacgaggaccgcaggggccagacagtcaatgccggtgtggctgagcggagctggctgtacctgaaaggatcatacatgcgctgtgaccgcaagatggaggtggcgttcatggtgtgtgccatcaacccttccattgacctgcacaccgactcgctggagcttctgcagctgcagcagaagctgctctggctgctctatgacctgggacatctggaaaggtaccccatggccttagggaacctggcagatctagaggagctggagcccacccctggccggccagacccactcaccctctaccacaagggcattgcctcagccaagacctactatcgggatgaacacatctacccctacatgtacctggctggctaccactgtcgcaaccgcaatgtgcgggaagccctgcaggcctgggcggacacggccactgtcatccaggactacaactactgccgggaagacgaggagatctacaaggagttctttgaagtagccaatgatgtcatccccaacctgctgaaggaggcagccagcttgctggaggcgggcgaggagcggccgggggagcaaagccagggcacccagagccaaggttccgccctccaggaccctgagtgcttcgcccacctgctgcgattctacgacggcatctgcaaatgggaggagggcagtcccacgcctgtgctgcacgtgggctgggccacctttcttgtgcagtccctaggccgttttgagggacaggtgcggcagaaggtgcgcatagtgagccgagaggccgaggcggccgaggccgaggagccgtggggcgaggaagcccgggaaggccggcggcggggcccacggcgggagtccaagccagaggagcccccgccgcccaagaagccagcactggacaagggcctgggcaccggccagggtgcagtgtcaggacccccccggaagcctcctgggactgtcgctggcacagcccgaggccctgaaggtggcagcacggctcaggtgccagcacccgcagcatcaccaccgccggagggtccagtgctcactttccagagtgagaagatgaagggcatgaaggagctgctggtggccaccaagatcaactcgagcgccatcaagctgcaactcacggcacagtcgcaagtgcagatgaagaagcagaaagtgtccacccctagtgactacactctgtctttcctcaagcggcagcgcaaaggcctctgaactactggggacttcggaccgcttgtggggacccaggctccgccttagtcccccaactctgagcccatgttctgcccccagcccaaaggggacaggcctcacctctacccaaaccctaggttcccggtcccgagtacagtctgtatcaaacccacgattttctccagctcagaacccagggctctgccccagtcgttagaatataggtctcttctcccagaatcccagccggccaatggaaacctcacgctgggtcctaattaccagtctttaaaggcccagcccctagaaacccaagctcctcctcggaaccgctcacctagagccagaccaacgttactcagggctcctcccagcttgtaggagctgaggtttcacccttaacccaagggagcacaggtcccacctccagcccggggagcctaggaccactcagcccctaggagtatatttccgcacttcagaattccatatcttgcgaatccaagctccctgccccaaataacttcagtcctgcttccagaatttggaaatcctagtttcctctccttcgtatcccgagtctgggacacaaaactccgcccccagcctatgagcatcctgagccccgccctcttcctgacgaaactggccccggatcagagcaggacctcccttccgaccctctgggaacctcccagaggtccagcccatctcggagcatcccggaggaaatctgcagaggggttaggagtgggtgacaagagcctgatctcttcctgttttgtacatagatttatttttcagttccaagaaagatgaatacattttgttaaaaaaaaaaaaaaaa

SEQ ID NO: 2 - protein, 610 aa, Homo sapiens
  human memin, multiple endocrine neoplasia-type 1(MEN1)
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGL

(19) United States
(12) Patent Application Publication
(10) Pub. No.: US 2008/0125389 A1
(43) Pub. Date: May 29, 2008

(54) Men1 is a marker and therapeutic target for breast and prostate cancer

(75) Inventors: Pamela L. Paris, San Francisco, CA (US); Colin C. Collins, San Rafael, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA Office of Technology Transfer, Oakland, CA (US)

(21) Appl. No.: 11/800,272

(22) Filed: May 3, 2007

Related U.S. Application Data
(60) Provisional application No. 60/798,066, filed on May 4, 2006.

(51) Int. Cl. A61K 31/70; C12Q 1/68; G01N 33/574; A61P 43/00; C12Q 1/02

(52) U.S. Cl. 514/44; 435/6; 435/7.23; 435/29

(57) ABSTRACT

The present invention provides genomic markers for determining the presence or absence of breast cancer. The invention further demonstrates that inhibition of MEN1 reduces breast or prostate cancer proliferation, thereby demonstrating that inhibition of MEN1 activity or expression can be used to reduce breast cancer or prostate cancer proliferation in an individual in need thereof.

### CROSS-REFERENCE TO RELATED PATENT APPLICATIONS

The present patent application claims benefit of priority to U.S. Provisional Patent Application No. 60/798,066, filed May 4, 2006, which is incorporated by reference in its entirety.

### STATEMENT AS TO RIGHTS TO INVENTIONS MADE UNDER FEDERALLY SPONSORED RESEARCH OR DEVELOPMENT

This invention was made with Government support under Grant Nos. P50 CA89520-05S1 and R01CA115484-01, awarded by the National Institutes of Health. The Government has certain rights in this invention.

### BACKGROUND OF THE INVENTION

Because of the importance of selecting appropriate treatment regimens for breast cancer patients, and for following their progress, the development of methods to monitor treatment as well as to sensitively diagnose breast cancer is of key interest to those in the medical community and their patients. Although available diagnostic procedures for breast cancer may be partially successful, the methods for detecting breast cancer and monitoring its treatment remain unsatisfactory. There is a critical need for diagnostic tests that can detect breast cancer at its early stages, when appropriate treatment may substantially increase the likelihood of positive outcome for the patient. It is also important to monitor breast cancer treatment to allow the treatment to be adapted as necessary to best serve the patient's clinical needs. Such diagnostic and monitoring methods will enable medical care professionals to identify breast cancer, select optimal treatment regimens for individual patients, and to assess the cancer before, during, and after treatment.

In addition, prostate cancer is the most commonly diagnosed non-cutaneous neoplasm among males in Western countries and is estimated to result in 28,900 deaths this year in the U.S. alone.

Determination of appropriate treatment for an individual cancer patient (e.g., having breast or prostate cancer) is complex with a wide variety of treatments and possible treatment combinations. Furthermore, while many treatments are effective, there remains a need for further therapies to address unmet needs of cancer patients.

### BRIEF SUMMARY OF THE INVENTION

The present invention provides methods for detecting the presence or absence of a breast cancer cell (e.g., in a breast biopsy or other biological sample) from an individual. In some embodiments, the methods comprise assaying the quantity of an MEN1 polynucleotide in the breast biopsy; and comparing the quantity with a control value representing the quantity of an MEN1 polynucleotide, wherein a higher amount in the assayed quantity in the breast biopsy compared to the control value indicates the presence of a breast cancer cell. In some embodiments, the MEN1 polynucleotide is an mRNA encoding MEN1. In some embodiments, the polynucleotide is chromosomal DNA. In some embodiments, the control value represents a quantity of MEN1 polynucleotide that distinguishes breast cancer cells from non-cancer cells. In some embodiments, the control value represents a quantity of MEN1 polynucleotide associated with the quantity found in a non-cancer cell. In some embodiments, the breast cancer is ductal carcinoma.

In some embodiments, the methods comprise assaying the quantity of an MEN1 polypeptide in the breast biopsy; and comparing the quantity with a control value representing the quantity of an MEN1 polypeptide, wherein a higher amount in the assayed quantity in the breast biopsy compared to the control value indicates the presence of a breast cancer cell. In some embodiments, the control value represents a quantity of MEN1 polypeptide that distinguishes breast cancer cells from non-cancer cells. In some embodiments, the control value represents a quantity of MEN1 polypeptide associated with the quantity found in a non-cancer cell. In some embodiments, the breast cancer is ductal carcinoma.

The present invention also provides methods of identifying an agent that inhibits breast cancer proliferation. In some embodiments, the methods comprise contacting a plurality of agents to a cell; selecting an agent that decreases expression or activity of an MEN1 polypeptide compared to expression or activity of MEN1 in the absence of the agent; and determining whether the selected agent inhibits breast cancer proliferation or metastasis, thereby identifying an agent that inhibits prostate cancer proliferation or metastasis. In some embodiments, the breast cancer is ductal carcinoma.

The present invention also provides methods of inhibiting proliferation of treating breast or prostate cancer cells. In some embodiments, the method comprises contacting the breast or prostate cancer cells with an agent that inhibits expression or activity of MEN1, thereby inhibiting proliferation of the breast or prostate cancer cells. In some embodiments, the cancer is breast cancer. In some embodiments, the cancer is prostate cancer. In some embodiments, the agent comprises an siRNA or antisense polynucleotide that inhibits MEN1 expression. In some embodiments, the agent comprises a polynucleotide encoding an siRNA or antisense polynucleotide that inhibits MEN1 expression.

### DEFINITIONS

An “MEN1 polynucleotide” refers to a polynucleotide encoding an MEN1 polypeptide (also referred to in the literature as “menin”). For example, an MEN1 polynucleotide can be an mRNA that encodes an MEN1 polypeptide or a chromosomal DNA sequence that comprises the gene sequences encoding the above-referenced mRNA. MEN1 polypeptide sequences are publicly available, e.g., in Genbank accession number U93237 (SEQ ID NO:2), displaying the human MEN1 sequence. See also WO 98/39439. An exemplary MEN1 cDNA sequence is also displayed in Genbank accession number U93237 (SEQ ID NO:1). A number of gene variants are known, some of which are described in, e.g., Agarwal (1997) Hum Mol Genet 6, 1169; Chandrasekharappa (1997) Science 276, 404; Lemmens (1997) Hum Mol Genet 6, 1177; Shimizu (1997) Jpn J Cancer Res 88, 1029; Aoki (1997) Jpn J Clin Oncol 27, 419; Mayr (1997) Eur J Endocrinol 137, 684 and are readily available on the internet. Probes for detection of MEN1 are available commercially from, e.g., Applied Biosystems Inc., Foster City, Calif. In some embodiments of the invention, the MEN1 polypeptides are at least 90, 95, 96, 97, 98, or 99% identical to SEQ ID NO:2. In some embodiments, the MEN1 polynucleotides of the invention comprise a polynucleotide sequence at least 90, 95, 96, 97, 98, or 99% identical to the coding sequence or the entire sequence of SEQ ID NO:1.

A “control value” refers to a value, typically pre-determined, that represents a numerical value or range of values associated with a phenotype. As used in the present application, control values represent a value or range (or one or more end points of a range) that represents: (1) MEN1 polynucleotide or polypeptide quantity or expression (or MEN1 chromosomal copy number) values associated with breast and/or prostate cancer cells, (2) MEN1 polynucleotide or polypeptide quantity or expression (or MEN1 chromosomal copy number) values associated with non-cancer cells (e.g., healthy cells), or (3) a value or range that distinguishes breast and/or prostate cancer cells from non-cancer cells. A “value” or “quantity” that “distinguishes” cancer and non-cancer cells, in the context of MEN1 expression or copy number, refers to a statistically determined value that represents a border between values typically associated with cancer and values typically associated with non-cancer cells. For example, where a majority of cancer cells have MEN1 expression values of 8-10, and a majority of non-cancer cells have MEN1 expression values of 1-4, then a value of 5-7 can be used to distinguish cancer and non-cancer cells in that values higher than 5-7 indicates the presence of cancer and values lower represent non-cancer cells. In many instances, there may be an overlap in values between cancer and non-cancer samples. In such situations, those of skill will recognize that a balance of false-positive and false-negative values can be determined in selected a preferred control value to distinguish between cancer and non-cancer cells.

The terms “tumor” or “cancer” in an animal (e.g., a human) refers to the presence of cells possessing characteristics such as atypical growth or morphology, including uncontrolled proliferation, immortality, metastatic potential, rapid growth and proliferation rate, and certain characteristic morphological features. Often, cancer cells will be in the form of a tumor, but such cells may also exist in isolation from one another within an animal. “Tumor” includes both benign and malignant neoplasms.

The terms “hybridizing specifically to”, “specific hybridization”, and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., a cell lysate or DNA preparation from a tissue biopy) A “stringent hybridization” and “stringent hybridization wash conditions” in the context of nucleic acid hybridization (e.g., as in array, Southern or northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell (2001) Molecular Cloning: A Laboratory Manual (3rd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra. for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

The term “nucleic acid” or “polynucleotide” as used herein refers to a deoxyribonucleotide or ribonucleotide in either single- or double-stranded form. The term encompasses nucleic acids containing known analogues of natural nucleotides which have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156).

The term “probe” or “nucleic acid probe”, as used herein, is defined to be one or more nucleic acid fragments whose specific hybridization to a sample can be detected. The probe may be unlabeled or labeled as described below so that its binding to the target or sample can be detected. The probe is produced from a source of nucleic acids from one or more particular (preselected) portions of a chromosome, e.g., one or more clones, an isolated whole chromosome or chromosome fragment, or a collection of polymerase chain reaction (PCR) amplification products. The probes of the present invention are produced from nucleic acids found in the regions described herein.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology (2003).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene (see, e.g., Bass, Nature, 411, 428-429 (2001); Elbashir et al., Nature, 411, 494-498 (2001); WO 00/44895; WO 01/36646; WO 99/32619; WO 00/01846; WO 01/29058; WO 99/07409; and WO 00/44914). “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferably about preferably about 20-30 base nucleotides, preferably about 20-25 nucleotides in length, e.g., 20, 21, 22, 23, 24, 25, 26, 27, 28, 29, or 30 nucleotides in length.

“Silencing” or “downregulation” refers to a detectable decrease of transcription and/or translation of a target sequence, i.e., the sequence targeted by the RNAi, or a decrease in the amount or activity of the target sequence or protein in comparison to the normal level that is detected in the absence of the interfering RNA or other nucleic acid sequence. A detectable decrease can be as small as at least 5% or at least 10%, or as great as 80%, 90% or 100%. More typically, a detectable decrease ranges from at least 20%, at least 30%, at least 40%, at least 50%, at least 60%, or at least 70%.

### BRIEF DESCRIPTION OF THE DRAWINGS

FIG. 1 illustrates TaqMan results for MEN1 in prostate cancer cell lines (PC3, DU145, LnCaP, MDAPCA2A) and a breast cancer cell line (BT474). For comparison, normal lung tissue was included which is known to weakly express MEN1. Results are presented as percent expression relative to the reference gene GUS.

FIGS. 2A-B illustrate demonstration of transfection efficiency in DU145 cells (A) and in BT474 cells (B) using a fluorescently labeled siRNA.

FIGS. 3A-B illustrate ACEA siRNA results in DU145 (A) and BT474 (B). The controls include Neg (cells grown in the presence of the transfection reagent) and Alexa Fluor (scramble siRNA). Cells are seeded at time 0, and transfected after 24 hrs. They are refed after 72 hrs. Data is normalized prior to transfection. As shown to the very right of FIG. 3A, from top to bottom, the lines represent Neg, AF, MEN12, MEN11, MEN13 and MEN14, respectively. As shown to the very right of FIG. 3B, from top to bottom, the lines represent Alexa Fluor, MEN2, Neg, MEN1, MEN3 and MEN4, respectively.

FIGS. 4A-B illustrate TaqMan results of RNA from siRNA experiments for DU145 (A) and BT474 (B). The controls include, Neg (cells grown in serum free media), HP (cells grown in the presence of the transfection reagent), and scramble siRNAs (AF and S5).

FIG. 5 illustrate cell counting experiments for DU145 cells grown in the presence of transfection reagent (NC—left bar) or transfected with either a scramble MEN1 siRNA (AF—middle bar) or an MEN1 siRNA (MEN4—right bar). Cells conditions are analogous to the ACEA and TaqMan experiments. Each experiment was run in duplicate. Fewer viable cells are present after MEN siRNA transfection.

### DETAILED DESCRIPTION OF THE INVENTION

### I. Introduction

The present invention is based in part on the discovery that the MEN1 gene acts as an oncogene in cancer cells and that inhibition of MEN1 expression results in a reduction in cancer cell proliferation and/or survival. This work therefore provides the first evidence that MEN1 acts to promote cancer cell growth and that inhibition of MEN1 expression or activity reduces cancer cell proliferation and/or survival.

Moreover, the inventors have discovered that MEN1 displayed a significant increase in expression and/or chromosomal copy number in breast cancer and prostate cancer cells, thereby showing that detection of increased expression of MEN1 indicates the presence of cancer cells in breast or prostate tissue. Thus, detection of increased expression of MEN1 (or increased chromosomal DNA copy number or MEN1 protein expression or stability) compared to the same criteria in healthy non-cancer cells is useful as a diagnostic and prognostic tool for breast and prostate cancer.

Typically diagnostic or prognostic methods will involve providing or isolating a biological sample from an individual (e.g., for detecting breast cancer, such samples include but are not limited to, a breast tissue biopsy or nipple discharge or other breast tissue sample) and detecting quantity of expression of MEN1 mRNA, MEN1 chromosomal DNA copy number, or MEN1 protein quantity or activity in the biological sample. The detected quantity or number is then compared to the corresponding value or range associated with a healthy sample. Alternatively, the detected quantity or number is compared to a value or range associated with a cancer tissue. In yet another alternative, the detected quantity or number is compared to a cut-off or threshold value that distinguishes between values typically found in healthy tissues and values typically found in cancerous tissues. Cut-off or threshold values can be generated or determined using publicly available statistical analysis software.

Detecting the quantity of expression of MEN1 mRNA, MEN1 chromosomal DNA copy number, or MEN1 protein quantity or activity in the biological sample can also be used to provide a prognosis or to assess the efficacy of an anti-cancer treatment, wherein the absence or reduction over time of MEN1 copy number or expression indicates that an individual is responding to the anti-cancer therapy.

Typically, the level of a polynucleotide or polypeptide of interest will be detected in, or isolated from, a biological sample. A “biological sample” refers to a cell or population of cells or a quantity of tissue or fluid from an animal. Most often, the sample has been removed from an animal, but the term “biological sample” can also refer to cells or tissue analyzed in vivo, i.e., without removal from the animal. Typically, a “biological sample” will contain cells from the animal, but the term can also refer to noncellular biological material, such as noncellular fractions of blood, saliva, or urine, that can be used to measure the cancer-associated MEN1 polynucleotide or polypeptide levels. Numerous types of biological samples can be used in the present invention, including, but not limited to, a tissue biopsy, a blood sample, a urine sample, or a nipple discharge.

Exemplary types of breast cancer that can be detected or treated according to the methods of the present invention include, e.g., ductal carcinoma, lobular carcinoma, inflammatory breast cancer, medullary carcinoma, mucinous carcinoma, Paget's Disease of the Nipple, phyllodes tumor, or tubular carcinoma.

### II. Detecting Chromosomal Regions or Parts Thereof

Genomic instability is a hallmark of solid tumors, and virtually no solid tumor exists that does not show some alterations of the genome. With the vast majority of tumors this instability is expressed at the level of the chromosomal complement, and thus is detectable by cytogenetic approaches (Mitelman, F., Catalog of Chromosome Aberrations in Cancer, 5th Edition (New York: Wiley-Liss) (1994)). However, aneuploidy or chromosomal rearrangement per se is not indicative of malignancy and many benign tumors can have an aberrant karyotype (Mitelman, 1994). To efficiently take advantage of chromosomal abnormalities as a marker, it is useful to know characteristic aberrations of the tumors that are to be differentiated.

As discussed in the examples, increased MEN1 chromosomal copy number and/or expression is found in prostate and breast cancer cells compared to normal cells. Accordingly, increased chromosomal copies of the MEN1 gene (e.g., coding sequences and/or upstream or downstream elements such as promoters including, but not limited to, nucleotides 5 kb upstream of the initiation of translation or transcription) may also be detected to diagnose (e.g., detect the presence or absence of) breast or prostate cancer.

Single or low-copy number probes that detect DNA within the genomic MEN1 locus are particularly useful for use in the invention. Various probes may be used to detect MEN1 copy number. An exemplary probe for MEN1 is BAC CTD-2220I9, commercially available at Invitrogen, Inc. (Carlsbad, Calif.).

Several techniques that permit the study of chromosomal complement are well known in the art. For example, fluorescence in-situ hybridization (FISH) can be used to study copy numbers of individual genetic loci or particular regions on a chromosome (Pinkel et al., Proc. Natl. Acad. Sci. U.S.A. 85, 9138-42 (1988)). Comparative genomic hybridization (CGH) (Kallioniemi et al. Science 258, 818-21 (1992)) may also be used (Houldsworth et al. Am J Pathol 145, 1253-60 (1994)) to probe for copy number changes of chromosomal regions as well as changes in chromosome number.

As appreciated by one of skill in the art, analysis of copy number can be performed using multiple probes to a particular chromosome or can be performed using a single probe, e.g., a centromeric probe, to detect change in copy number. Probes useful in the methods described here are available from a number of sources. For instance, P1 clones are available from the DuPont P1 library (Shepard, et al., Proc. Natl. Acad. Sci. USA, 92:2629 (1994), and available commercially from Genome Systems. Various libraries spanning entire chromosomes are also available commercially (Clonetech, South San Francisco, Calif.), or from the Los Alamos National Laboratory.

In one set of embodiments, the hybridizations are performed on a solid support. For example, probes that selectively hybridize to specific chromosomal regions can be spotted onto a surface. Conveniently, the spots are placed in an ordered pattern, or array, and the placement of the probes on the array is recorded to facilitate later correlation of results. The nucleic acid samples are then hybridized to the array. In one configuration, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958).

Arrays, particularly nucleic acid arrays can be produced according to a wide variety of methods well known to those of skill in the art (see, e.g., U.S. Pat. No. 6,040,138). For example, in a simple embodiment, “low density” arrays can simply be produced by spotting (e.g. by hand using a pipette) different nucleic acids at different locations on a solid support (e.g. a glass surface, a membrane, etc.).

This simple spotting approach has been automated to produce high density spotted arrays (see, e.g., U.S. Pat. No. 5,807,522). This patent describes the use of an automated systems that taps a microcapillary against a surface to deposit a small volume of a biological sample. The process is repeated to generate high density arrays. Arrays can also be produced using oligonucleotide synthesis technology. Thus, for example, U.S. Pat. No. 5,143,854 and PCT patent publication Nos. WO 90/15070 and 92/10092 teach the use of light-directed combinatorial synthesis of high density oligonucleotide arrays.

In another embodiment the array, particularly a spotted array, can include genomic DNA, e.g. overlapping clones that provide a high resolution scan of the amplicon corresponding to the region of interest. Amplicon nucleic acid can be obtained from, e.g., MACs, YACs, BACs, PACs, P1s, cosmids, plasmids, inter-Alu PCR products of genomic clones, restriction digests of genomic clone, cDNA clones, amplification (e.g., PCR) products, and the like.

In various embodiments, the array nucleic acids are derived from previously mapped libraries of clones spanning or including the target sequences of the invention, as well as clones from other areas of the genome, as described below. The arrays can be hybridized with a single population of sample nucleic acid or can be used with two differentially labeled collections (as with a test sample and a reference sample).

Target elements of various sizes, ranging from 1 mm diameter down to 1 μm can be used. Smaller target elements containing low amounts of concentrated, fixed probe DNA are used for high complexity comparative hybridizations since the total amount of sample available for binding to each target element will be limited. Thus it is advantageous to have small array target elements that contain a small amount of concentrated probe DNA so that the signal that is obtained is highly localized and bright. Such small array target elements are typically used in arrays with densities greater than 10⁴/cm². Relatively simple approaches capable of quantitative fluorescent imaging of 1 cm² areas have been described that permit acquisition of data from a large number of target elements in a single image (see, e.g., Wittrup, Cytometry 16: 206-213, 1994).

Arrays on solid surface substrates with much lower fluorescence than membranes, such as glass, quartz, or small beads, can achieve much better sensitivity. Substrates such as glass or fused silica are advantageous in that they provide a very low fluorescence substrate, and a highly efficient hybridization environment. Covalent attachment of the target nucleic acids to glass or synthetic fused silica can be accomplished according to a number of known techniques. Nucleic acids can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). Quartz cover slips, which have at least 10-fold lower autofluorescence than glass, can also be silanized.

Alternatively, the samples can be placed in separate wells or chambers and hybridized in their respective well or chambers. The art has developed robotic equipment permitting the automated delivery of reagents to separate reaction chambers, including “chip” and microfluidic techniques, which allow the amount of the reagents used per reaction to be sharply reduced. Chip and microfluidic techniques are taught in, for example, U.S. Pat. No. 5,800,690, Orchid, “Running on Parallel Lines” New Scientist, Oct. 25, 1997, McCormick, et al., Anal. Chem. 69:2626-30 (1997), and Turgeon, “The Lab of the Future on CD-ROM?” Medical Laboratory Management Report. December 1997, p. 1. Automated hybridizations on chips or in a microfluidic environment are contemplated methods of practicing the invention.

Although microfluidic environments are one embodiment of the invention, they are not the only defined spaces suitable for performing hybridizations in a fluid environment. Other such spaces include standard laboratory equipment, such as the wells of microtiter plates, Petri dishes, centrifuge tubes, or the like can be used.

In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. The preferred size range is from about 200 bp to about 1000 bases, more preferably between about 400 to about 800 bp for double stranded, nick translated nucleic acids.

In some applications it is necessary to block the hybridization capacity of repetitive sequences. Thus, in some embodiments, human genomic DNA or Cot-1 DNA is used to block non-specific hybridization.

In Comparative Genomic Hybridization (CGH) methods, a first collection of (sample) nucleic acids (e.g. from a possible tumor) is labeled with a first label, while a second collection of (control) nucleic acids (e.g. from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc. In some embodiments, the hybridization protocol of Pinkel et al. (1998) Nature Genetics 20:207-211 or of Kallioniemi (1992) Proc. Natl. Acad Sci USA 89:5321-5325 (1992) is often used.

In general, there is a tradeoff between hybridization specificity (stringency) and signal intensity. Thus, in some embodiments, the wash is performed at the highest stringency that produces consistent results and that provides a signal intensity greater than approximately 10% of the background intensity. Thus, in some embodiments, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In some embodiments, background signal is reduced by the use of a detergent (e.g., C-TAB) or a blocking reagent (e.g., sperm DNA, cot-1 DNA, etc.) during the hybridization to reduce non-specific binding. The hybridization may be performed, for example, in the presence of about 0.1 to about 0.5 mg/ml DNA (e.g., cot-1 DNA). The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background membranes can be used (see, e.g., Chu (1992) Electrophoresis 13:105-114). The sensitivity for detection of spots (“target elements”) of various diameters on the candidate membranes can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., membranes, glass, fused silica) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

Other nucleic acid hybridization formats are also known to those skilled in the art. Such formats are described, for example in Sambrook and Russell, supra. These includes analyses such as Southern blotting. The sensitivity of the hybridization assays may also be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Ploidy, i.e., chromosome number, may also be determined using quantitative PCR such as real-time PCR (see, e.g., Suzuki et al., Cancer Res. 60:5405-9 (2000)). For example, quantitative microsatellite analysis (QuMA) can be performed for rapid measurement of relative DNA sequence copy number. In QuMA, the copy number of a test locus relative to a pooled reference is assessed using quantitative, real-time PCR amplification of loci carrying simple sequence repeats. Use of simple sequence repeats is advantageous because of the large numbers that are mapped precisely.

Additional protocols for quantitative PCR are provided in Innis et al. (1990) PCR Protocols, A Guide to Methods and Applications, Academic Press, Inc. N.Y.).

### III. Detecting Gene Expression

As described above, it has been discovered that increased expression of MEN1 in a breast or prostate sample compared to a non-cancer control indicates the presence of cancerous cells in the sample. Accordingly, the present invention provides for methods of detecting expression of MEN1, including expression of mRNAs or proteins encoded by the genes.

In one embodiment, the presence of cancer is evaluated by determining the level of expression of mRNA encoding MEN1. Methods of evaluating RNA expression of a particular gene are well known to those of skill in the art, and include, inter alia, hybridization and amplification based assays.

### Direct Hybridization-Based Assays

Methods of detecting and/or quantifying the level of MEN1 gene transcripts (mRNA or cDNA made therefrom) using nucleic acid hybridization techniques are known to those of skill in the art. For example, one method for evaluating the presence, absence, or quantity of MEN1 polynucleotides involves a northern blot. Gene expression levels can also be analyzed by techniques known in the art, e.g., dot blotting, in situ hybridization, RNase protection, probing DNA microchip arrays, and the like.

### Amplification-Based Assays

In another embodiment, amplification-based assays are used to measure the expression level of MEN1. In such an assay, the nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the level of expression of the gene of interest in the sample. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). The known MEN1 nucleic acid sequences are sufficient to enable one of skill to routinely select primers to amplify any portion of the gene. Exemplary sequences for the MEN1 cDNAs can be found in, e.g., Genbank accession number U93236.

In one embodiment, a TaqMan™ based assay is used to quantify MEN1 polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

### Production of Antibodies and Immunological Detection

Polypeptides encoded by the genes described herein can be detected and/or quantified by any methods known to those of skill in the art. Samples can be from any biological source, including e.g., tissue (e.g., breast or prostate) biopsies, tumors, and bodily fluids such as blood, urine, semen, etc.

In some embodiments, antibodies can also be used to detect polypeptides, or fragments thereof (e.g., at least 10, 15, 20, 25, or more contiguous amino acids) of the MEN1 polypeptide. Antibodies to these polypeptides can be produced using well known techniques (see, e.g., Harlow & Lane, Antibodies: A Laboratory Manual (1988) and Harlow & Lane, Using Antibodies (1999); Coligan, Current Protocols in Immunology (1991); Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Such antibodies are typically used for diagnostic or prognostic applications, e.g., in the detection of prostate or breast cancer, including ductal carcinomas.

Polypeptides of the invention or a fragment thereof may be used to produce antibodies specifically reactive with the polypeptide. For example, a recombinant MEN1 polypeptide or an antigenic fragment thereof, may be isolated. Recombinant protein is a useful immunogen for the production of monoclonal or polyclonal antibodies. Alternatively, a synthetic peptide derived from MEN1 polypeptide sequences and conjugated to a carrier protein can be used as an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Once specific antibodies are available, binding interactions with the MEN1 polypeptide can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra).

Immunoassays also often use a labeling agent to specifically bind to and label the complex formed by the antibody and antigen. The labeling agent may itself be one of the moieties comprising the antibody/antigen complex. Thus, the labeling agent may be a labeled polypeptide or a labeled antibody that binds the protein of interest. Alternatively, the labeling agent may be a third moiety, such as a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the labeling agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Commonly used assays include noncompetitive assays, e.g., sandwich assays, and competitive assays. In competitive assays, the amount of polypeptide present in the sample is measured indirectly by measuring the amount of a known, added (exogenous) polypeptide of interest displaced (competed away) from an antibody that binds by the unknown polypeptide present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels, enzymes (e.g., horse radish peroxidase, alkaline phosphatase and others commonly used in an ELISA), and calorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize the polypeptide of interest, or secondary antibodies that recognize an antibody that binds the polypeptide.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Methods of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### IV. Inhibition of MEN1 to Treat or Reduce Breast or Prostate Cancer

Inhibitors of MEN1 polypeptides are useful for treating cancer, including breast and prostate cancer. For example, administration of the MEN1 inhibitors can be used to treat breast or prostate cancer or at least reduce the progression or symptoms of breast or prostate cancer.

MEN1 inhibitors can inhibit MEN1 polypeptide activity (e.g., DNA binding activity or ability to interact with other protein components), MEN1 polypeptide expression, MEN1 mRNA expression or MEN1 mRNA stability.

As discussed in the examples, inhibition of MEN1 expression using siRNAs resulted in reduced breast cancer or prostate cancer cell proliferation. Accordingly, in some embodiments, siRNAs or antisense polynucleotides are used to inhibit MEN1 mRNA stability or expression, either in vitro or in vivo, to reduce or inhibit breast or prostate cancer proliferation. This approach may utilize, for example, siRNA and/or antisense oligonucleotides to block transcription or translation of a specific mutated mRNA, either by inducing degradation of the mRNA with a siRNA or by masking the mRNA with an antisense nucleic acid.

siRNA

Double stranded siRNA that corresponds to the MEN1 gene transcript can be used to silence the transcription and/or translation of MEN1 by inducing degradation of MEN1 mRNA transcripts, and thus inhibit proliferation of breast or prostate cancer cells. In some embodiments, the siRNA is about 5 to about 100 nucleotides in length, more typically about 10 to about 50 nucleotides in length, most typically about 15 to about 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002). siRNA nucleic acids may comprise naturally occurring nucleotides or modified nucleotides such as, e.g., phosphorothioate, methylphosphonate, and -anomeric sugar-phosphate, backbone-modified nucleotides.

siRNA can be delivered to the subject using any means known in the art, including by injection, inhalation, or oral ingestion of the siRNA. Another suitable delivery system for siRNA is a colloidal dispersion system such as, for example, macromolecule complexes, nanocapsules, microspheres, beads, and lipid-based systems including oil-in-water emulsions, micelles, mixed micelles, and liposomes. An exemplary colloidal system of this invention is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art. In addition, siRNA polynucleotides can be delivered using a recombinant expression vector (e.g., a viral vector based on an adenovirus, a herpes virus, a vaccinia virus, or a retrovirus). Examples of successful in vivo delivery of siRNA polynucleotides are described in, e.g., Reich et al., Mol Vis 9:210-6 (2003); Dom et al., Nucleic Acids Res 32(5):e49 (2004); Li, et al., Nat Med 11(9):944-51 (2005); Song et al. Nat Med 9(3):347-51 (2003); Zender, et al. Proc Natl Acad Sci USA 100(13):7797-802 (2003); Soutschek et al., Nature 432(7014):173-8 (2004); Urban-Klein, et al., Gene Ther 12(5):461-6 (2005); Pal, et al. Int J Oncol 26(4):1087-91 (2005); Morrissey, et al. Nat Biotechnol 23(8):1002-7 (2005); Schiffelers et al. Nucleic Acids Res 32(19):e149 (2004); Lu, et al. Adv Genet 54:117-42 (2005).

### Antisense Oligonucleotides

Antisense oligonucleotides that specifically hybridize to nucleic acid sequences encoding MEN1 polypeptides can also be used to silence the transcription and/or translation of MEN1, and thus treat or inhibit prostate or breast cancer cell proliferation. Antisense nucleic acids are DNA or RNA molecules that are complementary to at least a portion of a specific mRNA molecule (see, e.g., Weintraub, Scientific American, 262:40 (1990)). Typically, synthetic antisense oligonucleotides are generally between 15 and 25 bases in length. Antisense nucleic acids may comprise naturally occurring nucleotides or modified nucleotides such as, e.g., phosphorothioate, methylphosphonate, and -anomeric sugar-phosphate, backbone-modified nucleotides.

In the cell, the antisense nucleic acids hybridize to the corresponding mRNA, forming a double-stranded molecule. The antisense nucleic acids, interfere with the translation of the mRNA, since the cell will not translate a mRNA that is double-stranded. In some embodiments, antisense oligomers of about 15 nucleotides are used. The use of antisense methods to inhibit the translation of genes is well known in the art (Marcus-Sakura, Anal. Biochem., 172:289, (1988)). Less commonly, antisense molecules which bind directly to the DNA may be used.

Delivery of antisense polynucleotides specific for MEN1 genes can be achieved using any means known in the art including, e.g., direct injection, inhalation, or ingestion of the polynucleotides. In addition, antisense polynucleotides can be delivered using a recombinant expression vector (e.g., a viral vector based on an adenovirus, a herpes virus, a vaccinia virus, or a retrovirus) or a colloidal dispersion system (e.g., liposomes) as described herein.

### V. Identification of Inhibitors of Genes and Polypeptides of the Invention

Further inhibitors of MEN1 expression or activity can be identified using standard screening assays. Screening assays may involve, for example, screening libraries of molecules for their ability to inhibit MEN1 expression or activity or by identifying agents that inhibit prostate or breast cancer proliferation (wherein the breast cancer or prostate cancer cell proliferation can be inhibition via inhibition of MEN1, e.g., using an siRNA as described herein) and then determining that the mechanism of action of the identified agent involves inhibition of MEN1 expression or activity.

### A. Agents that Modulate Polypeptides Described Herein

The agents tested as modulators of polypeptides of the invention can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). Modulators also include agents designed to modulate (increase or decrease) the level of mRNA encoding polypeptide (e.g., antisense molecules, ribozymes, DNAzymes, small inhibitory RNAs (siRNAs) and the like) or the level of translation from an mRNA (e.g., translation blockers such as an antisense molecules that are complementary to translation start or other sequences on an mRNA molecule).

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### B. Methods of Screening for Modulators of the Polypeptides of the Invention

A number of different screening protocols can be utilized to identify agents that modulate the level of expression or activity of an MEN1 polynucleotide or MEN1 polypeptide of the invention. In some embodiments, such assays are performed on cells, particularly mammalian cells, and especially human cells. In general terms, the screening methods involve screening a plurality of agents to identify an agent that modulates the activity of a polypeptide of the invention by, e.g., binding to the polypeptide, preventing an activator from binding to the polypeptide, increasing association of an inhibitor with the polypeptide, or inhibiting expression of the polypeptide or mRNA encoding MEN1.

Any cell expressing a full-length MEN1 polypeptide or an active fragment or variant thereof can be used to identify modulators. In some embodiments, the cells are eukaryotic cells lines transformed to express a heterologous MEN1 polypeptide.

1. Polypeptide Binding Assays

Preliminary screens can be conducted by screening for agents capable of binding to MEN1 polypeptides, as at least some of the agents so identified are likely modulators of a polypeptide of the invention. Binding assays are also useful, e.g., for identifying endogenous proteins that interact with the polypeptides described herein. For example, antibodies or other molecules that bind polypeptides of the invention can be identified in binding assays. Binding assays can involve, but are not limited to, use of isolated polypeptides, crude extracts, or cell-based assays.

Binding assays can involve contacting a polypeptide with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound the polypeptide or displacement of labeled substrates. The polypeptides used in these assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind to the polypeptide when expressed together in a host cell.

2. Polypeptide Activity

Polypeptide activity can be assessed using a variety of in vitro and in vivo assays to determine functional, chemical, and physical effects to identify modulators.

In some embodiments, for example, agents are screened for the ability to inhibit binding of MEN1 to JunD. See, e.g., Agarwel et al., Hum. Molec. Genet. 6:1169-1175 (1997). Alternatively, agents are screened for the ability to inhibit binding of MEN1 to SMAD3, or suppression of TGFB-induced and SMAD3-induced transcriptional activity. See, e.g., Kaji et al., Proc. Nat. Acad. Sci. 98:3837-3842 (2001).

Samples or assays that are treated with a potential inhibitor or activator (e.g., a “test compound”) are compared to control samples without the test compound, to examine the extent of modulation. Control samples (untreated with candidate compounds are assigned a relative activity value of 100. Inhibition of the polypeptides of the invention is achieved when the activity value relative to the control is less than about 90%, optionally less than 50%, optionally less than 25-1%.

3. Expression Assays

Screening assays for a compound that modulates the expression of polynucleotides and polypeptides described herein are also provided. Screening methods generally involve conducting cell-based assays in which test compounds are contacted with one or more cells expressing one or more polypeptide of the invention, and then detecting an increase or decrease in expression (either transcript or translation product). Assays can be performed with any cells that express a polypeptide.

Expression can be detected in a number of different ways. As described herein, the expression level of a polynucleotide can be determined by probing the mRNA expressed in a cell with a probe that specifically hybridizes with an encoded transcript (or complementary nucleic acid derived therefrom). Alternatively, a polypeptide can be detected using immunological methods, e.g., an assay in which a cell lysate is probed with antibodies that specifically bind to the polypeptide.

Reporter systems can also be used to identify modulators of expression. A variety of different types of cells can be utilized in promoter reporter assays. Cells that do not endogenously express a particular polypeptide of interest can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the HEK293, HepG2, COS, CHO and HeLa cell lines. In some embodiments, expression of a report gene under the control of the MEN1 is monitored.

Various controls can be conducted to ensure that an observed activity is authentic including running parallel reactions with cells that lack the reporter construct or by not contacting a cell harboring the reporter construct with test compound. Compounds can also be further validated as described below.

4. Validation

Agents that are initially identified by any of the foregoing screening methods can be further tested to validate the activity.

In some embodiments, the MEN1 inhibitors are tested for their ability to reduce or inhibit breast or prostate cell culture growth or proliferation.

Validity of the inhibitors, for example, can also be tested in suitable animal models. The basic format of such methods involves administering a lead compound identified during an initial screen to an animal that serves as a model for human disease (e.g., prostate cancer or breast cancer) and/or determining if expression or activity of a polypeptide or polynucleotide of interest is in fact modulated.

### C. Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

A molecule of interest (e.g., a polypeptide or polynucleotide, or a modulator thereof) can be bound to the solid-state component, directly or indirectly, via covalent or non-covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

The invention provides in vitro assays for identifying, in a high throughput format, compounds that can modulate the expression or activity of the genes or polypeptides of the invention. Control reactions that measure polypeptide activity in a cell in a reaction that does not include a potential modulator are optional, as the assays are highly uniform. Such optional control reactions are appropriate and increase the reliability of the assay. Accordingly, in some embodiments, the methods of the invention include such a control reaction. For each of the assay formats described, “no modulator” control reactions that do not include a modulator provide a background level of binding activity.

### VI. Pharmaceutical Formulation and Administration

Inhibitors of MEN1 can be administered directly to a mammalian subject (e.g., a human) using any route known in the art, including e.g., by injection (e.g., intravenous, intraperitoneal, subcutaneous, intramuscular), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part a of prepared food or drug.

The dose administered to a patient, in the context of the present invention should be sufficient to effect a beneficial response in the subject over time, e.g., at least a reduction of prostate or breast cancer cell growth, proliferation or metastasis. The optimal dose level for any patient will depend on a variety of factors including the efficacy of the specific modulator employed, the age, body weight, physical activity, and diet of the patient, on a possible combination with other drugs, and on the cancer. The size of the dose also will be determined by the existence, nature, and extent of any adverse side-effects that accompany the administration of a particular compound or vector in a particular subject.

### VII. Kits

For use in diagnostic and research applications, kits are also provided by the invention. The kits of the invention may comprise any or all of the reagents to perform the methods described herein. In the diagnostic and research applications such kits may include any or all of the following: assay reagents, buffers, nucleic acids that bind to at least one of the genomic regions or genes described herein, hybridization probes and/or primers, antibodies or other moieties that specifically bind to at least one of the polypeptides encoded by the genes described herein, etc. Kits may optionally include a device (e.g., a syringe) for extracting a breast or prostate tissue biopsy.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLE

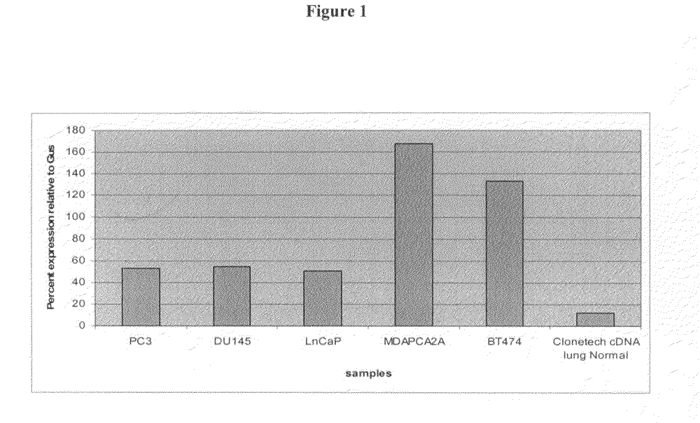

We reported the association of copy number gain at chromosome 11q13.1 and risk of post-prostatectomy reoccurrence in patients with prostate cancer. See, Paris, P. L. et al. Hum Mol Genet 13:1303-13 (2004). Our TaqMan studies implicated the menin (MEN1) gene at this locus. Since menin is historically known as a tumor suppressor gene in multiple endocrine neoplasia (Pannett, A. A. & Thakker, R. V. Endocr Relat Cancer 6:449-73 (1999); Chandrasekharappa, S. C. & Teh, B. T. J Intern Med 253:606-15 (2003)), we sought to provide in vitro phenotype data demonstrating MEN1 as an oncogene in prostate cancer. Copy number data was available in our laboratory for the breast cancer cell line, BT474, which is derived from a ductal carcinoma. The aCGH data was queried and shown to have copy number gain for the MEN1 locus. Therefore, BT474 was included with 3 commercially available prostate cancer cell lines, PC3, DU145 and LnCaP as well as one obtained through collaboration, MDAPCA2A. All 5 cell lines showed higher levels of MEN1 expression than the lung tissue control, which expresses MEN1 at low levels (Diehn, M. et al. Nucleic Acids Res 31:219-23 (2003) (FIG. 1). The highest MEN1 expressers were BT474 and MDAPCA2A.

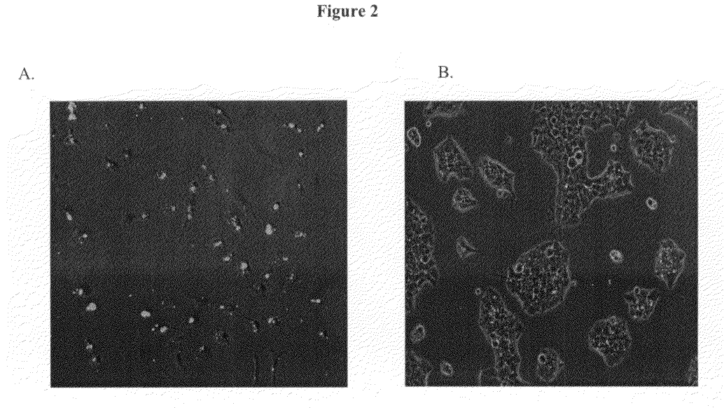

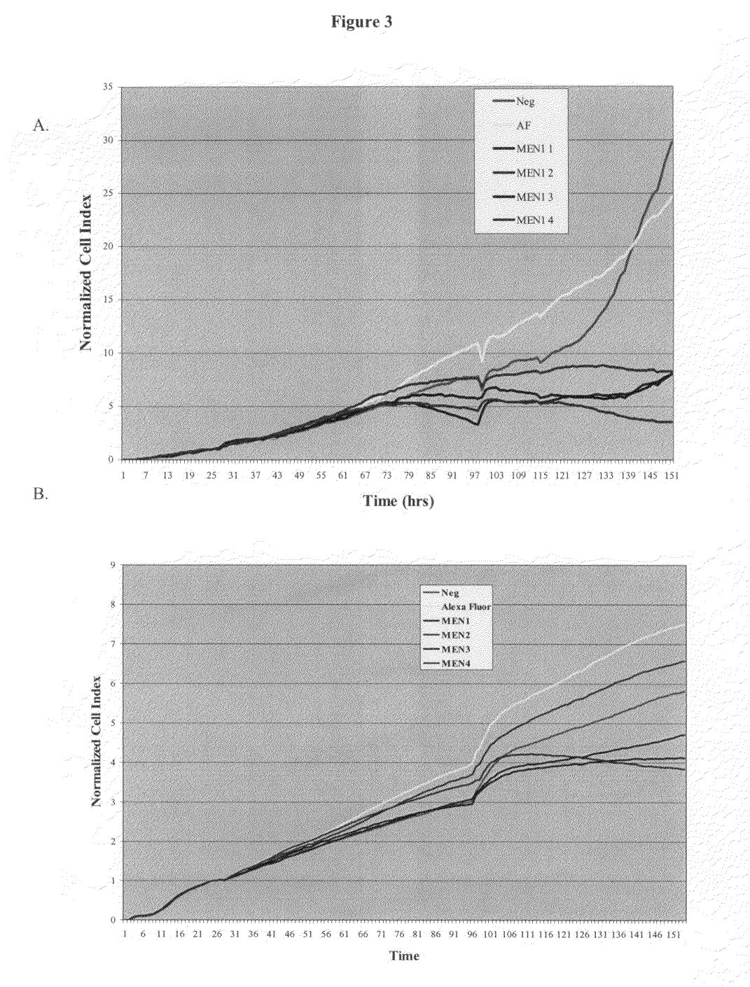

LnCaP and MDAPCA2A are semi-adherent cell lines and are not readily amenable to gene knock down assays. We chose to pursue the adherent cell lines DU145 and BT474 with siRNAs designed to target MEN1. Four siRNAs designed to target MEN1 were purchased from Qiagen. Transfection efficiency was visually assessed with a fluorescently labeled scramble siRNA in both DU145 and BT474 (FIG. 2). We used the ACEA instrument for the in vitro phenotype measurements. The ACEA instrument allows real-time phenotype measurements with a microelectronic cell sensor system that is integrated into the bottom of microtiter plates. The amount of impedence measured correlates to cell index. FIG. 3 shows the siRNA ACEA results for both DU145 and BT474. These experiments have been replicated several times. Both cell lines showed decreased cell growth with siRNAs targeting MEN1. MEN1 siRNA#2 does not show a phenotype in BT474 cells. Interestingly, a SNP maps to siRNA MEN2.

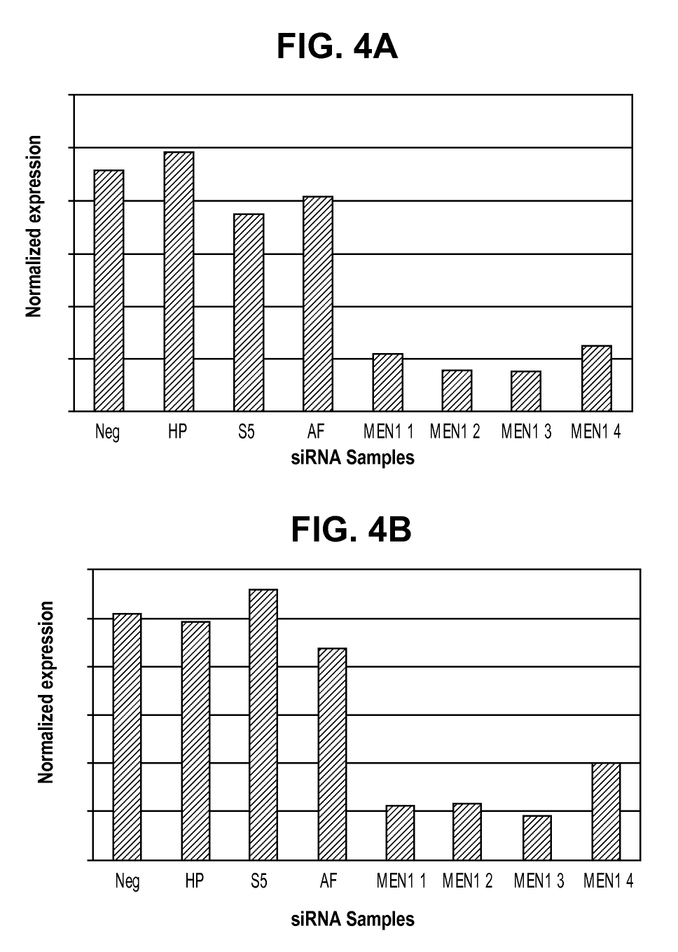

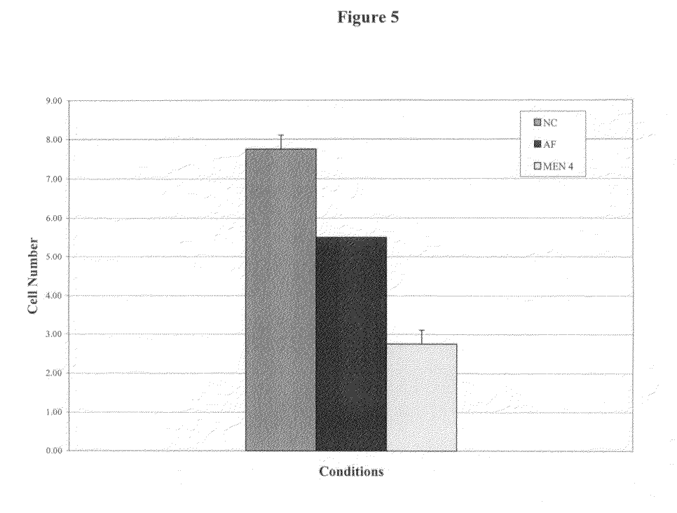

In addition, we have isolated RNA from these siRNA cell line experiments and have performed TaqMan assays to provide independent MEN1 knockdown validation. All cell lines exposed to MEN1 siRNAs showed decreased MEN1 expression (FIG. 4). Therefore, the observed phenotype is linked to MEN1. DU145 cell counting experiments, conducted under conditions analagous to the ACEA and TaqMan experiments, showed a decreased cell index when exposed to MEN1 siRNA#4 (FIG. 5). FACs analysis and expression array studies are underway to gather additional phenotype data and pathway insight.

We have therefore demonstrated in vitro that MEN1 acts as a oncogene in both prostate and breast cancer cell lines. This supports our previous copy number and expression studies in prostate cancer and provides data that MEN1 is important in breast cancer as well.

We conclude that MEN1 is a therapeutic target in both prostate and breast cancer. These new findings also show that detection of copy number or expression from the MEN1 locus can be used as a diagnostic target not only in prostate cancer, but in breast cancer as well.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING

SEQ ID NO:1 - Exemplary MEN1 cDNA sequence

| | |
|---|---|
| 1 | ggtgtccgga gccgcggacc tagagatccc agaagccaca gcgcagcggc ccggcccgcc |
| 61 | actatttcca ggctctgcgg ggcaggggcc gccgcccacc gcccgccgcc atggggctga |
| 121 | aggccgccca gaagacgctg ttcccgctgc gctccatcga cgacgtggtg cgcctgtttg |
| 181 | ctgccgagct gggccgagag gagccggacc tggtgctcct ttccttggtg ctgggcttcg |
| 241 | tggagcattt tctggctgtc aaccgcgtca tccctaccaa cgttcccgag ctcaccttcc |
| 301 | agcccagccc cgcccccgac ccgcctggcg gcctcaccta ctttcccgtg gccgacctgt |
| 361 | ctatcatcgc cgccctctat gcccgcttca ccgcccagat ccgaggcgcc gtcgacctgt |
| 421 | ccctctatcc tcgagaaggg ggtgtctcca gccgtgagct ggtgaagaag gtctccgatg |
| 481 | tcatatggaa cagcctcagc cgctcctact tcaaggatcg ggcccacatc cagtccctct |
| 541 | tcagcttcat cacaggcacc aaattggaca gctccggtgt ggcctttgct gtggttgggg |
| 601 | cctgccaggc cctgggtctc cgggatgtcc acctcgccct gtctgaggat catgcctggg |
| 661 | tagtgtttgg gcccaatggg gagcagacag ctgaggtcac ctggcacggc aagggcaacg |
| 721 | aggaccgcag gggccagaca gtcaatgccg gtgtggctga gcggagctgg ctgtacctga |
| 781 | aaggatcata catgcgctgt gaccgcaaga tggaggtggc gttcatggtg tgtgccatca |
| 841 | acccttccat tgacctgcac accgactcgc tggagcttct gcagctgcag cagaagctgc |
| 901 | tctggctgct ctatgacctg ggacatctgg aaaggtaccc catggcctta gggaacctgg |
| 961 | cagatctaga ggagctggag cccacccctg gccggccaga cccactcacc ctctaccaca |
| 1021 | agggcattgc ctcagccaag acctactatc gggatgaaca catctacccc tacatgtacc |
| 1081 | tggctggcta ccactgtcgc aaccgcaatg tgcgggaagc cctgcaggcc tgggcggaca |
| 1141 | cggccactgt catccaggac tacaactact gccgggaaga cgaggagatc tacaaggagt |
| 1201 | tctttgaagt agccaatgat gtcatcccca acctgctgaa ggaggcagcc agcttgctgg |
| 1261 | aggcgggcga ggagcggccg ggggagcaaa gccagggcac ccagagccaa ggttccgccc |
| 1321 | tccaggaccc tgagtgcttc gcccacctgc tgcgattcta cgacggcatc tgcaaatggg |
| 1381 | aggagggcag tcccacgcct gtgctgcacg tgggctgggc cacctttctt gtgcagtccc |
| 1441 | taggccgttt tgagggacag gtgcggcaga aggtgcgcat agtgagccga gaggccgagg |
| 1501 | cggccgaggc cgaggagccg tggggcgagg aagcccggga aggccggcgg cggggcccac |
| 1561 | ggcgggagtc caagccagag gagcccccgc cgcccaagaa gccagcactg gacaagggcc |
| 1621 | tgggcaccgg ccagggtgca gtgtcaggac ccccccggaa gcctcctggg actgtcgctg |
| 1681 | gcacagcccg aggccctgaa ggtggcagca cggctcaggt gccagcaccc gcagcatcac |
| 1741 | caccgccgga gggtccagtg ctcactttcc agagtgagaa gatgaagggc atgaaggagc |
| 1801 | tgctggtggc caccaagatc aactcgagcg ccatcaagct gcaactcacg gcacagtcgc |
| 1861 | aagtgcagat gaagaagcag aaagtgtcca cccctagtga ctacactctg tctttcctca |
| 1921 | agcggcagcg caaaggcctc tgaactactg gggacttcgg accgcttgtg gggacccagg |
| 1981 | ctccgcctta gtcccccaac tctgagccca tgttctgccc ccagcccaaa ggggacaggc |
| 2041 | ctcacctcta cccaaaccct aggttcccgg tcccgagtac agtctgtatc aaacccacga |
| 2101 | ttttctccag ctcagaaccc agggctctgc cccagtcgtt agaatatagg tctcttctcc |
| 2161 | cagaatccca gccggccaat ggaaacctca cgctgggtcc taattaccag tctttaaagg |
| 2221 | cccagcccct agaaacccaa gctcctcctc ggaaccgctc acctagagcc agaccaacgt |
| 2281 | tactcagggc tcctcccagc ttgtaggagc tgaggtttca cccttaaccc aagggagcac |
| 2341 | aggtcccacc tccagcccgg ggagcctagg accactcagc ccctaggagt atatttccgc |
| 2401 | acttcagaat tccatatctt gcgaatccaa gctccctgcc ccaaataact tcagtcctgc |
| 2461 | ttccagaatt tggaaatcct agtttcctct ccttcgtatc ccgagtctgg gacacaaaac |
| 2521 | tccgccccca gcctatgagc atcctgagcc ccgccctctt cctgacgaaa ctggccccgg |
| 2581 | atcagagcag gacctccctt ccgaccctct gggaacctcc cagaggtcca gcccatctcg |
| 2641 | gagcatcccg gaggaaatct gcagaggggt taggagtggg tgacaagagc ctgatctctt |
| 2701 | cctgttttgt acatagattt atttttcagt tccaagaaag atgaatacat tttgttaaaa |
| 2761 | aaaaaaaaaa aa |

SEQ ID NO:2 - Exemplary MEN1 polypeptide sequence
MGLKAAQKTLFPLRSIDDVVRLFAAELGREEPDLVLLSLVLGFV
EHFLAVNRVIPTNVPELTFQPSPAPDPPGGLTYFPVADLSIIAALYARFTAQIRGAVD
LSLYPREGGVSSRELVKKVSDVIWNSLSRSYFKDRAHIQSLFSFITGTKLDSSGVAFA
VVGACQALGLRDVHLALSEDHAWVVFGPNGEQTAEVTWHGKGNEDRRGQTVNAGVAER
SWLYLKGSYMRCDRKMEVAFMVCAINPSIDLHTDSLELLQLQQKLLWLLYDLGHLERY
PMALGNLADLEELEPTPGRPDPLTLYHKGIASAKTYYRDEHIYPYMYLAGYHCRNRNV
REALQAWADTATVIQDYNYCREDEEIYKEFFEVANDVIPNLLKEAASLLEAGEERPGE
QSQGTQSQGSALQDPECFAHLLRFYDGICKWEEGSPTPVLHVGWATFLVQSLGRFEGQ
VRQKVRIVSREAEAAEAEEPWGEEAREGRRRGPRRESKPEEPPPPKKPALDKGLGTGQ
GAVSGPPRKPPGTVAGTARGPEGGSTAQVPAPAASPPPEGPVLTFQSEKMKGMKELLV
ATKINSSAIKLQLTAQSQVQMKKQKVSTPSDYTLSFLKRQRKGL

What is claimed is:

1. A method for detecting the presence or absence of a breast cancer cell in a breast biopsy from an individual, the method comprising,
assaying the quantity of an MEN1 polynucleotide in the breast biopsy; and
comparing the quantity with a control value representing the quantity of an MEN1 polynucleotide, wherein a higher amount in the assayed quantity in the breast biopsy compared to the control value indicates the presence of a breast cancer cell.

2. The method of claim 1, wherein the MEN1 polynucleotide is an mRNA encoding MEN1.

3. The method of claim 1, wherein the polynucleotide is chromosomal DNA.

4. The method of claim 1, wherein the control value represents a quantity of MEN1 polynucleotide that distinguishes breast cancer cells from non-cancer cells.

5. The method of claim 1, wherein the control value represents a quantity of MEN1 polynucleotide associated with the quantity found in a non-cancer cell.

6. The method of claim 1, wherein the breast cancer is ductal carcinoma.

7. A method for detecting the presence or absence of a breast cancer cell in a breast biopsy from an individual, the method comprising,
assaying the quantity of an MEN1 polypeptide in the breast biopsy; and
comparing the quantity with a control value representing the quantity of an MEN1 polypeptide, wherein a higher amount in the assayed quantity in the breast biopsy compared to the control value indicates the presence of a breast cancer cell.

8. The method of claim 7, wherein the control value represents a quantity of MEN1 polypeptide that distinguishes breast cancer cells from non-cancer cells.

9. The method of claim 7, wherein the control value represents a quantity of MEN1 polypeptide associated with the quantity found in a non-cancer cell.

10. The method of claim 7, wherein the breast cancer is ductal carcinoma.

11. A method of identifying an agent that inhibits breast cancer proliferation, the method comprising,
contacting a plurality of agents to a cell;
selecting an agent that decreases expression or activity of an MEN1 polypeptide compared to expression or activity of MEN1 in the absence of the agent; and
determining whether the selected agent inhibits breast cancer proliferation or metastasis, thereby identifying an agent that inhibits prostate cancer proliferation or metastasis.

12. The method of claim 11, wherein the breast cancer is ductal carcinoma.

13. A method of inhibiting proliferation of treating breast or prostate cancer cells, the method comprising,
contacting the breast or prostate cancer cells with an agent that inhibits expression or activity of MEN1, thereby inhibiting proliferation of the breast or prostate cancer cells.

14. The method of claim 13, wherein the cancer is breast cancer.

15. The method of claim 13, wherein the cancer is prostate cancer.

16. The method of claim 13, wherein the agent comprises an siRNA or antisense polynucleotide that inhibits MEN1 expression.

17. The method of claim 13, wherein the agent comprises a polynucleotide encoding an siRNA or antisense polynucleotide that inhibits MEN1 expression.
